(19) United States
(12) Patent Application Publication
(10) Pub. No.: US 2024/0225754 A1
(43) Pub. Date: Jul. 11, 2024

(54) CONNECTOR AND RELEASE MECHANISMS FOR SURGICAL DEVICES AND KITS

(71) Applicant: CAIRA Surgical, New York, NY (US)

(72) Inventors: Jody Lee CLAYPOOL, Warsaw, IN (US); Gordon L. GOODCHILD, West Palm Harbor, FL (US); Joseph WHITLEY, Leesburg, IN (US)

(21) Appl. No.: 18/611,348

(22) Filed: Mar. 20, 2024

Related U.S. Application Data
(63) Continuation-in-part of application No. 18/124,554, filed on Mar. 21, 2023.
Application No. 18/124,554 is a continuation-in-part of application No. 18/069,338, filed on Dec. 21, 2022.
Application No. 18/069,338 is a continuation-in-part of application No. 17/901,475, filed on Sep. 1, 2022, now Pat. No. 11,896,319.
Application No. 17/901,475 is a continuation of application No. 17/017,015, filed on Sep. 10, 2020, now Pat. No. 11,432,882.
Application No. 17/017,015 is a continuation-in-part of application No. 16/573,095, filed on Sep. 17, 2019.
(60) Provisional application No. 63/491,527, filed on Mar. 21, 2023.
(60) Provisional application No. 63/491,450, filed on Mar. 21, 2023.

(51) Int. Cl. A61B 34/20; A61B 34/10; A61B 90/00

(52) U.S. Cl. CPC A61B 34/20; A61B 2034/108; A61B 2034/2051; A61B 2090/3916; A61B 2090/3975; A61B 2090/3991

(57) ABSTRACT

Connectors and release mechanisms are provided. Connectors can include a distally positioned locking stud with a tapered head portion and an array of coupling studs. The locking stud can exhibit an axis of symmetry along its length. The array of coupling studs can extend radially outward in a plane orthogonal to the axis of symmetry of the locking stud. A connector socket can include a coupling socket configured to receive one of the coupling studs when the coupling socket moves along a first direction, and a hinged release mechanism. The release mechanism can be configured to oppose a biasing force that is directed opposite the first direction. An application of a release force on the release mechanism in the direction of the biasing force can release the engagement of the connector and connector socket and cause the coupling socket to move opposite the first direction.

### CROSS-REFERENCES TO RELATED APPLICATIONS

This application claims the priority and benefit of U.S. Provisional Application No. 63/491,527, filed on Mar. 21, 2023, which is hereby incorporated by reference in its entirety; this application also claims the priority and benefit of U.S. Provisional Application No. 63/491,450, filed on Mar. 21, 2023, which is also hereby incorporated by reference in its entirety. Further, this application is a continuation-in-part of U.S. patent application Ser. No. 18/124,554, entitled “Systems and Methods for Medical Object Tracking in Obstructed Environments” and having Docket No. 0421.0001-CIP-CIP2 filed on Mar. 21, 2023, which is a continuation in-part of U.S. patent application Ser. No. 18/069,338, entitled “Systems and Methods for Medical Object Tracking in Obstructed Environments” and having Docket No. 0421.0001-CIP-CIP1 filed on Dec. 21, 2022, which is a continuation in-part of U.S. patent application Ser. No. 17/901,475, entitled “System and Method for Medical Object Tracking,” filed Sep. 1, 2022, now U.S. Pat. No. 11,896,319, which is a continuation of U.S. patent application Ser. No. 17/017,015, entitled “System and Method for Medical Object Tracking,” filed Sep. 10, 2020, now U.S. Pat. No. 11,432,882, which is a continuation in-part of U.S. patent application Ser. No. 16/573,095, entitled “System and Method for Medical Object Tracking,” filed Sep. 17, 2019, the contents of all of which are also hereby incorporated by reference in their entirety.

### FIELD

Materials and components consistent with the present disclosure are directed to connectors and release mechanisms, particularly connectors and release mechanisms for use with surgical devices and kits.

### BACKGROUND

Surgical devices and kits can include connectors and release mechanism between components that are not intuitive and that require a user to apply force to a release mechanism that is in a direction opposite to the direction that separates the two components. Further complicating the disassociation of components with hand-operated release mechanisms, a user's hands in a surgical environment can be gloved and possibly wet, making manipulation of hand-operated release mechanisms difficult.

### SUMMARY

In one aspect, embodiments consistent with the present disclosure include a surgical device or component with a connector. In embodiments, the surgical device or component can exhibit an axis such that the connector is situated along the axis in a distal relationship with a functional portion of the surgical device or component. In embodiments, the surgical device or component can further include a handgrip portion that encloses the axis, where the handgrip portion lies between the connector and the functional portion of the surgical device or component. In an embodiment, the connector can include a distally positioned locking stud with a tapered head portion, and an array of coupling studs between the locking stud and the handgrip portion. In an embodiment, the locking stud can further exhibit a narrow neck portion between the tapered head portion and the array of coupling studs, the narrow neck portion being adjacent the tapered head portion, the locking stud further exhibiting an axis of symmetry along its length, the axis of symmetry being aligned with the axis of the surgical device or component. Further still, in an embodiment, the array of coupling studs can include four coupling studs situated such that two of the four coupling studs share a first axis of symmetry along their length, and an other two of the four coupling studs share a second axis of symmetry along their length, the first axis of symmetry forming a right angle with the second axis of symmetry, and the four coupling studs extending radially outward from a point where the first axis of symmetry intersects the second axis of symmetry. Further still, in an embodiment, the first axis of symmetry and the second axis of symmetry can define a plane such that the axis of the surgical device or component forms a right angle to the plane.

In a further aspect, embodiments consistent with the present disclosure include a surgical kit, which can include a handheld probe with a pointer structure, a handgrip portion, and a connector. In embodiments, the handheld probe can exhibit an axis enclosed by the handgrip portion, where the connector is situated along the handheld probe axis in a distal relationship with the pointer structure such that the handgrip portion lies between the pointer structure and the connector. In an embodiment, the connector can include a distally positioned locking stud with a tapered head portion, and an array of coupling studs between the locking stud and the handgrip portion. In an embodiment, the locking stud can further exhibit a narrow neck portion between the tapered head portion and the array of coupling studs, the narrow neck portion being adjacent the tapered head portion, the locking stud further exhibiting an axis of symmetry along its length, the axis of symmetry being aligned with the handheld probe axis. Further still, in an embodiment, the array of coupling studs can include four coupling studs situated such that two of the four coupling studs share a first axis of symmetry along their length, and an other two of the four coupling studs share a second axis of symmetry along their length, the first axis of symmetry forming a right angle with the second axis of symmetry, and the four coupling studs extending radially outward from a point where the first axis of symmetry intersects the second axis of symmetry. Further still, in an embodiment, the first axis of symmetry and the second axis of symmetry can define a plane such that the handheld probe axis forms a right angle to the plane.

In another aspect, an embodiment can include any of the previous embodiments, and further include a second surgical component with a connector socket. In an embodiment, the connector socket can include a coupling socket configured to receive at least one of the four coupling studs when the coupling socket moves along a first direction relative to the at least one of the four coupling studs, a partial collar structure, and a release mechanism hinged to the paddle body with a movable terminal edge in a distal relationship to the hinge. In an embodiment, the release mechanism can be configured to oppose a biasing force that is directed opposite the first direction. Further still, in an embodiment, the partial collar structure can be configured to engage the narrow neck portion of the distally positioned locking stud adjacent the tapered head portion when the at least one of the four coupling studs is received in the coupling socket in the first direction and the movable terminal edge of the release mechanism is adjacent a surface of the tapered head. Further still, in an embodiment, an application of a release force on the release mechanism in the direction of the biasing force can release the engagement of the partial collar structure with the narrow neck portion and cause the coupling socket to move opposite the first direction.

In another aspect, an embodiment can include the previous embodiment where the second surgical component is a paddle attachment with a paddle body.

In other aspects, an embodiment can include any of the previous embodiments: (a) where the tapered head portion exhibits a narrowing head circumference in a direction away from the handheld grip portion; (b) where the hinge is a living hinge; (c) where each of the four coupling studs exhibit a cylindrical shape; (d) where the application of the release force causes the paddle attachment to disengage from the surgical device or component or handheld probe in the direction of the release force; (e) where, when the partial collar structure engages the narrow neck portion of the distally positioned locking stud adjacent the tapered head portion, and when the movable terminal edge of the release mechanism is adjacent a surface of the tapered head, the movable terminal edge of the release mechanism is configured to apply a force along the axis of symmetry of the locking stud in the direction of the surgical device or component or handheld probe; (f) where the release mechanism is a button release; (g) where the movable terminal edge is between the hinge and the coupling socket; (h) where the surgical device or component, or handheld probe with the pointer structure, is configured to enable registration of points of interest on a patient; and/or (i) where the second surgical component, or paddle attachment, in combination with the surgical device or component, or handheld probe, is configured to enable navigation of other surgical instruments, such as surgical cutting jigs.

In a further aspect, embodiments consistent with this disclosure can include a surgical kit including a paddle device with a paddle body and a connector socket. In an embodiment, the connector socket can include a coupling socket configured to receive a coupling stud when the coupling socket moves along a first direction relative to the coupling stud, a partial collar structure, and a release mechanism hinged to the paddle body with a movable terminal edge in a distal relationship to the hinge. In an embodiment, the release mechanism can be configured to oppose a biasing force that is directed opposite the first direction. Further still, in an embodiment, the partial collar structure can be configured to engage a narrow neck portion of a distally-positioned locking stud adjacent a tapered head portion on the locking stud when the coupling stud is received in the coupling socket in the first direction and the movable terminal edge of the release mechanism is adjacent a surface of the tapered head portion, and where an application of a release force on the release mechanism in the direction of the biasing force releases the engagement of the partial collar structure with the narrow neck portion and causes the coupling socket to move opposite the first direction.

Additional features and embodiments of the invention will be set forth in part in the description which follows, and in part will be obvious from the description, or may be learned by practice of the invention. It is to be understood that both the foregoing general description and the following detailed description are exemplary and explanatory only and are not restrictive of the claimed subject matter.

### BRIEF DESCRIPTION OF THE DRAWINGS

The accompanying drawings, which are incorporated in and constitute a part of this specification, illustrate exemplary embodiments and together with the description, serve to explain the principles of the disclosure. In the figures:

FIG. 1 depicts a handheld probe and a close-up view of a connector consistent with this disclosure;

FIG. 2 depicts a two views of connector socket and paddle attachment consistent with this disclosure; and

FIG. 3 depicts the connector socket of FIG. 2 engaged with the connector of FIG. 1 consistent with this disclosure.

### DESCRIPTION OF THE EMBODIMENTS

Reference will now be made in detail to the disclosed embodiments of the disclosure, examples of which are illustrated in the accompanying drawings. Wherever possible, the same reference numbers will be used throughout the drawings to refer to the same or like parts.

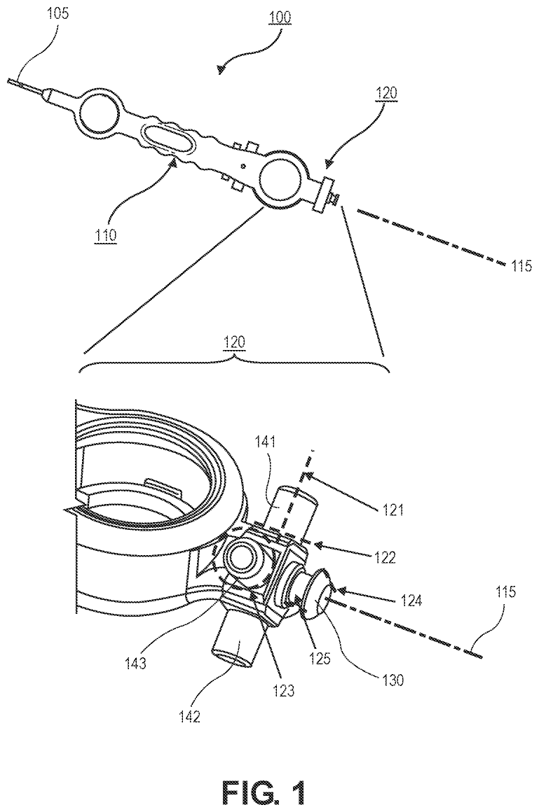

FIG. 1 depicts components of a surgical kit consistent with this disclosure. For example, handheld probe 100 with connector 120 is depicted at the top of FIG. 1, with a close up view of connector 120 at the bottom of FIG. 1. Features of handheld probe 100 can include pointer structure 105 and handgrip portion 110. Consistent with this disclosure, handheld probe 100 can include an axis 115 which runs through handheld probe 100, including within the portion enclosed by handgrip portion 110. One of ordinary skill in the art would appreciate that handheld probe 100 can be configured for use with a surgical navigation system. For example, handheld probe 100 can be configured to enable registration of certain points on a patient utilizing the position of the pointer structure 105 on handheld probe 100.

Consistent with this disclosure, handheld probe 100 can also include connector 120, where connector 120 is situated along the handheld probe axis 115 in a distal relationship with the pointer structure 105 such that the handgrip portion 110 lies between the pointer structure 105 and the connector 120.

Although FIG. 1 depicts handheld probe 100 with connector 120, embodiments consistent with this disclosure can include any surgical device or component with connector 120, where the surgical device or component generally exhibits an axis 115 such that the connector 120 is situated along the axis 115 in a distal relationship with a functional portion of the surgical device or component. Further still, the surgical device or component can include a handgrip portion 110 that encloses the axis 115, where the handgrip portion 110 lies between the connector 120 and the functional portion of the surgical device or component.

Consistent with this disclosure, the connector 120 can include a distally positioned locking stud 130 with a tapered head portion 124. Connector 120 can also include an array of coupling studs (where coupling stud 141, coupling stud 142, and coupling stud 143 are shown, and a fourth coupling stud—positioned opposite coupling stud 143—is not shown). As depicted in FIG. 1, consistent with this disclosure, the array of coupling studs lie between the locking stud 130 and the handgrip portion 110.

As depicted in FIG. 1, consistent with this disclosure, the locking stud 130 further exhibits a narrow neck portion 125 between the tapered head portion 124 and the array of coupling studs. As depicted, the narrow neck portion 125 is adjacent the tapered head portion 124, the locking stud 130 further exhibiting an axis of symmetry 115 along its length, the axis of symmetry 115 also being the handheld probe axis 115.

As shown in FIG. 1, the array of coupling studs can include four coupling studs (where coupling stud 141, coupling stud 142, and coupling stud 143 are shown) situated such that two of the four coupling studs (i.e., coupling stud 141 and coupling stud 142) share a first axis of symmetry 121 along their length, and an other two of the four coupling studs (where coupling stud 143 is shown, and another coupling stud, not shown, lies opposite coupling stud 143) share a second axis of symmetry along their length, the first axis of symmetry 121 forming a right angle with the second axis of symmetry, and the four coupling studs extending radially outward from a point where the first axis of symmetry 121 intersects the second axis of symmetry. Circumference 123 of a coupling stud (such as, for example, coupling stud 143), and which is a function of a diameter of the coupling stud, can be selected (in connection with the selection of a diameter of a coupling socket, described below) to control the amount of play in an attachment device, described further below.

Further still, as is apparent from FIG. 1, consistent with this disclosure, the first axis of symmetry 121 and the second axis of symmetry define a plane such that the axis 115 forms a right angle to that plane.

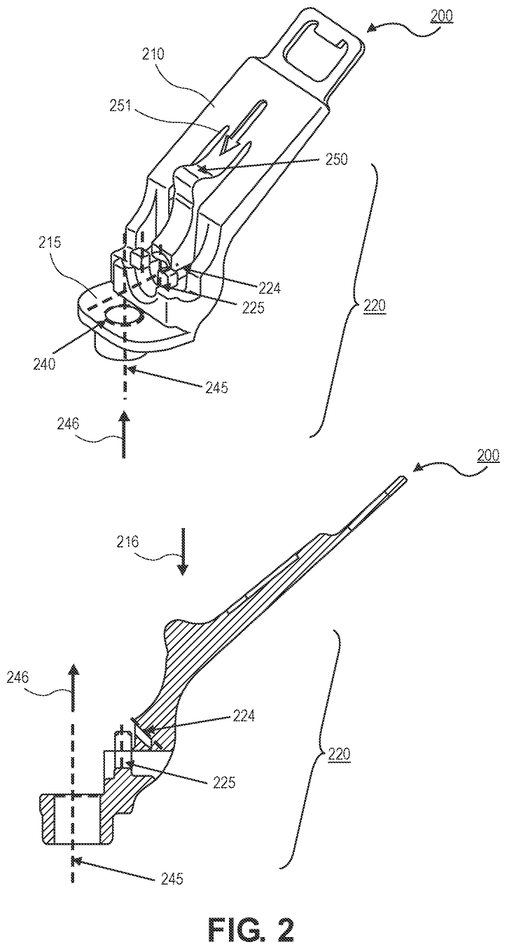

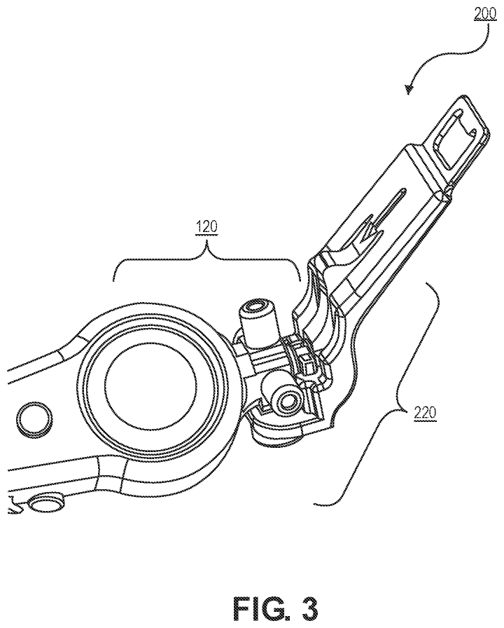

FIG. 2 depicts two views of another component of a surgical kit consistent with this disclosure: paddle attachment 200. Consistent with this disclosure, paddle attachment 200 can include paddle body 210 and a connector socket 220. The connect socket 220 can include a coupling socket 240 configured to receive at least one of the four coupling studs (i.e., coupling stud 141, coupling stud 142, coupling stud 143, or the coupling stud not shown in FIG. 1) when the coupling socket 240 moves along a first direction 246 relative to the at least one of the four coupling studs. Connector socket 220 can also include a partial collar structure 225 and a release mechanism 250 hinged to the paddle body 210. Release mechanism 250 can also include a movable terminal edge 224 in a distal relationship to the hinge 251. Consistent with this disclosure, the release mechanism 250 is configured to oppose a biasing force 216 that is directed opposite the first direction 246. For reference FIG. 3 depicts the connector socket 220 of FIG. 2 engaged with the connector 120 of FIG. 1.

As shown in FIGS. 2 and 3, the partial collar structure 225 is configured to engage the narrow neck portion 125 of the distally positioned locking stud 130 adjacent the tapered head portion 124 when one of the four coupling studs is received in the coupling socket 240 (the coupling socket moving in the first direction 246 relative to a coupling stud) and the movable terminal edge 224 of the release mechanism 250 is adjacent a surface of the tapered head 124. When engaged, axis 215 in FIG. 2 aligns with axis 115 of FIG. 1.

Consistent with this disclosure, an application of a release force in the direction 216 on the release mechanism 250 releases the engagement of the partial collar structure 225 with the narrow neck portion 125 and causes the coupling socket 240 to move opposite the first direction 246 (i.e., the direction 216). Reference 245 indicates an axis within coupling socket 240 that is aligned with direction 246.

One of ordinary skill in the art would appreciate that the position of the axes of symmetry of the coupling studs (such as the first axis of symmetry 121 associated with coupling studs 141 and 142) establishes a rotation position for the paddle attachment 200, where the axes of symmetry of the coupling studs are perpendicular relative to the long axis 115 of the handheld probe 100.

Further still, the distance 122 shown in FIG. 1 establishes a position along the long axis of the handheld probe 100 for the paddle attachment 200.

In addition, as described above, the diameter of a coupling stud (associated with, for example, circumference 123 of coupling stud 143) and the diameter of the coupling socket 240 can be selected to control the amount of play between the paddle attachment 200 and the handheld probe 100. This feature, in combination with the orientation of the axes of symmetry of the coupling studs, can control rotational position, and perpendicularity, of the paddle attachment 200 relative to the long axis 115 of the handheld probe 100.

One of ordinary skill in the art would also appreciate that the position of the tapered head portion 124 and the movable edge 224 (in combination with the engagement of the partial collar structure 225 and the narrow neck portion 125) provides locking and a constant force along the axis 115.

Further still, the hinge 251 associated with the release mechanism 250 can be a living hinge integrated into the paddle attachment 200, which can allow for tightly controlled tolerancing with no additional parts.

Although FIGS. 2 and 3 depict paddle attachment 200 with connector socket 220, embodiments consistent with this disclosure can include any second surgical component with connector socket 220, where the second surgical component can be used in combination with the surgical device or component with axis 115 and handgrip portion 110, such as, but not limited to, facilitating or enabling the registration or navigation of other surgical devices.

The dimensions and scales depicted in FIGS. 1-3 are examples applicable to one possible embodiment of the invention. Other embodiments have different scales and sizes.

Further still, although four coupling studs have been illustrated in FIG. 1 with connector 120, one of ordinary skill in the art would appreciate that connector 120 can include fewer than four (such as one, two, or three coupling studs), or more than four coupling studs (such as five, six, seven, eight, or more coupling studs) consistent with this disclosure. Further still, while the individual axis of symmetry of each coupling stud (such as axis 121) may be aligned in a plane that is orthogonal to an axis 115 associated with a functional aspect of a surgical device or component with connector 120, the arrangement of a set of axes of symmetry of multiple coupling studs can be, but does not have to be, arranged to lie symmetrically in the plane orthogonal to axis 115. For example, connector 120 of FIG. 1 is described as having four coupling studs (141, 142, 143, and a fourth not shown) with axes of symmetry (such as axis 121) distributed at 90-degree intervals in a plane orthogonal to axis 115. Consistent with this disclosure, there can fewer than four coupling studs, and more than four coupling studs distributed in a symmetrical fashion (such as two studs oriented at 180 degrees with respect to each other, three studs oriented at 120 degrees with respect to each other, five studs oriented at 72 degrees with respect to each other, six studs oriented at 60 degrees with respect to each other, etc.). Further still, consistent with this disclosure, there can be any number of coupling studs distributed in an asymmetrical fashion according to a need, or preferable orientation, of the attachment with the connector socket 220 with respect to the surgical device or component with connector 120. For example, connector 120 can include a single coupling stud with a single axis 121. Alternatively, connector 120 can include two or more coupling studs arranged such that the two or more axis (each of which is associated with a coupling stud) is arranged in an asymmetrical manner in a plane orthogonal to axis 115.

The examples of embodiments and implementations described above may vary in details and terms without departing from the scope of the invention.

Other embodiments of the invention will be apparent to those skilled in the art from consideration of the specification and practice of the embodiments disclosed herein. It is intended that this specification and examples be considered as examples only, with a true scope and spirit of the invention being indicated by the following claims.

What is claimed is:

1. A surgical kit comprising:
a first component including:
a handgrip portion; and
a connector
wherein the first component exhibits a component axis enclosed by the handgrip portion and the connector is situated at a terminal portion of the component axis;
wherein the connector comprises:
a distally positioned locking stud with a tapered head portion; and
an array of coupling studs between the locking stud and the handgrip portion;
wherein the locking stud further exhibits a narrow neck portion between the tapered head portion and the array of coupling studs, the narrow neck portion being adjacent the tapered head portion, the locking stud further exhibiting an axis of symmetry along its length, the axis of symmetry being aligned with the component axis;
wherein the array of coupling studs comprise four coupling studs situated such that two of the four coupling studs share a first axis of symmetry along their length, and an other two of the four coupling studs share a second axis of symmetry along their length, the first axis of symmetry forming a right angle with the second axis of symmetry, and the four coupling studs extending radially outward from a point where the first axis of symmetry intersects the second axis of symmetry; and
wherein the first axis of symmetry and the second axis of symmetry define a plane such that the component axis forms a right angle to the plane.

2. The surgical kit of claim 1, further comprising:
a second component with a component body and connector socket;
wherein the connector socket comprises:
a coupling socket configured to receive at least one of the four coupling studs when the coupling socket moves along a first direction relative to the at least one of the four coupling studs;
a partial collar structure; and
a release mechanism hinged to the component body with a movable terminal edge in a distal relationship to the hinge;
wherein the release mechanism is configured to oppose a biasing force that is directed opposite the first direction;
wherein the partial collar structure is configured to engage the narrow neck portion of the distally positioned locking stud adjacent the tapered head portion when the at least one of the four coupling studs is received in the coupling socket in the first direction and the movable terminal edge of the release mechanism is adjacent a surface of the tapered head; and
wherein an application of a release force on the release mechanism in the direction of the biasing force releases the engagement of the partial collar structure with the narrow neck portion and causes the coupling socket to move opposite the first direction.

3. The surgical kit of claim 2, wherein the tapered head portion exhibits a narrowing head circumference in a direction away from the handheld grip portion.

4. The surgical kit of claim 2, wherein the hinge is a living hinge.

5. The surgical kit of claim 2, wherein each of the four coupling studs exhibit a cylindrical shape.

6. The surgical kit of claim 2, wherein the application of the release force causes the second component to disengage from the first component in the direction of the release force.

7. The surgical kit of claim 2, wherein, when the partial collar structure engages the narrow neck portion of the distally positioned locking stud adjacent the tapered head portion, and when the movable terminal edge of the release mechanism is adjacent a surface of the tapered head, the movable terminal edge of the release mechanism is configured to apply a force along the axis of symmetry of the locking stud in the direction of the first component.

8. The surgical kit of claim 2, wherein the release mechanism is a button release.

9. The surgical kit of claim 2, wherein the movable terminal edge is between the hinge and the coupling socket.

10. The surgical kit of claim 1, wherein the first component is a handheld probe with a pointer structure such that the connector is situated along the component axis in a distal relationship with the pointer structure and the handgrip portion lies between the pointer structure and the connector.

11. The surgical kit of claim 2, wherein the second component is a paddle attachment and the component body is a paddle body.

12. The surgical kit of claim 11, wherein the first component is a handheld probe with a pointer structure such that the connector is situated along the component axis in a distal relationship with the pointer structure and the handgrip portion lies between the pointer structure and the connector.

13. The surgical kit of claim 12, wherein the handheld probe with the pointer structure is configured to enable registration of points of interest on a patient.

14. The surgical kit of claim 12, wherein the paddle attachment, in combination with the handheld probe, is configured to enable navigation of surgical cutting jigs.

15. A surgical kit comprising:
a surgical component, including:
a component body; and
a connector socket;
wherein the connector socket comprises:
a coupling socket configured to receive a coupling stud when the coupling socket moves along a first direction relative to the coupling stud;
a partial collar structure; and
a release mechanism hinged to the component body with a movable terminal edge in a distal relationship to the hinge;
wherein the release mechanism is configured to oppose a biasing force that is directed opposite the first direction;
wherein the partial collar structure is configured to engage a narrow neck portion of a distally-positioned locking stud adjacent a tapered head portion on the locking stud when the coupling stud is received in the coupling socket in the first direction and the movable terminal edge of the release mechanism is adjacent a surface of the tapered head portion; and
wherein an application of a release force on the release mechanism opposite the biasing force releases the engagement of the partial collar structure with the narrow neck portion and causes the coupling socket to move opposite the first direction.

16. The surgical kit of claim 15, wherein the surgical component is a paddle attachment and the component body is a paddle body.

17. The surgical kit of claim 15, wherein the hinge is a living hinge.

18. The surgical kit of claim 15, wherein the coupling stud exhibits a cylindrical shape.

19. The surgical kit of claim 15, wherein the release mechanism is a button release.

20. The surgical kit of claim 15, wherein the movable terminal edge is between the hinge and the coupling socket.
